(19) United States
(12) Patent Application Publication
(10) Pub. No.: US 2015/0374331 A1
(43) Pub. Date: Dec. 31, 2015

(54) ULTRASONIC PROBE

(71) Applicants: Woo-jin CHO, (US); ALPINION MEDICAL SYSTEMS CO., LTD., Hwaseong-si Gyeonggi-do (KR)

(72) Inventors: Woo-jin CHO, Seongnam-si (KR); Wonho NOH, Seoul (KR); Sang-woong LEE, Yongin-Si (KR); Sang-seok LEE, Gyeongju-si (KR); Jaewon LEE, Incheon (KR); Wonse PARK, Seoul (KR)

(73) Assignees: ALPINION MEDICAL SYSTEMS CO., LTD., Hwaseong-si, Gyeonggi-do (KR); Woo-jin CHO, Seongnam-si, Gyeonggi-do (KR)

(21) Appl. No.: 14/766,684

(22) PCT Filed: Feb. 7, 2013
(86) PCT No.: PCT/KR2013/000972

§ 371 Date: Aug. 7, 2015

(51) Int. Cl. A61B 8/12; A61B 8/00

(52) U.S. Cl. CPC A61B 8/12; A61B 8/4455; A61B 8/4272

(57) ABSTRACT

Embodiments of the present disclosure provide an ultrasonic cavity probe including a grip configured to be held by a user, a lens unit having a predetermined curvature radius, and configured to be inserted into a bodily cavity and to be brought into contact with a skin inside the bodily cavity, a head unit including a first side for mounting the lens unit and rounded corners, and a connecting portion configured to connect the head unit and the grip and to make a first angle with the head unit.

### TECHNICAL FIELD

The present disclosure relates to an ultrasonic probe, and more particularly, to an ultrasonic probe of an insertion type for inspecting general bodily cavities including ear, nostril, sublingual, tonsil and throat.

### BACKGROUND

The statements in this section merely provide background information related to the present disclosure and do not necessarily constitute prior art.

In recent years, an ultrasonic medical device is widely used in hospitals or in general internal medicine clinics to perform an ultrasound inspection.

The ultrasound inspection is a method of inspecting abnormality of tissue using an ultrasonic wave, in which a specific site inside the body is irradiated with the ultrasonic wave, and a reflected ultrasonic wave from the site is analyzed to determine a possible existence of an abnormal tissue. It is mainly used to diagnose a lesion tissue, such as a tumor, or a fetus. In order to perform such ultrasonic examination, an ultrasonic probe is employed to irradiate the tissue or the like with the ultrasonic wave, to thereby diagnose an abnormality of tissue or the like.

The principle of emitting the ultrasonic wave by the ultrasonic probe is generally using a piezoelectric member embedded in the probe, from which an ultrasonic wave is generated. The emitted ultrasonic wave passes through an acoustic matching layer and an acoustic lens, and then a target object to be inspected is irradiated with the ultrasonic wave. The acoustic lens serves to focus the ultrasonic wave traveling forward to a specific site, by which a focal length of the ultrasonic wave can be determined.

Otolaryngology clinics adopt a linear probe having a focal length of 20 millimeters in performing an extra mandible tonsil inspection or the like.

In order to obtain an accurate image in inspecting the tonsil region or the like, the ultrasonic probe needs to be inserted into the bodily cavity to perform the tonsil inspection or the like in the bodily cavity.

However, currently used linear probes are too bulky to be inserted into the cavity. Besides, the linear probe has too long a focal length, and hence it is hard to obtain a sharp image even if the tonsil inspection is performed by inserting the linear probe into the bodily cavity.

In addition, such a linear probe is inappropriate for use with a nose or a back area of an ear and other narrow and curved sites of the body.

### DISCLOSURE

### Technical Problem

An ultrasonic probe having a slim shape is desired to perform physical inspections by inserting the probe directly into bodily cavities including tonsil, sublingual, and other such sites.

Further, an ultrasonic cavity probe having a new structure or shape is desired, which allows an ultrasonic wave to be focused at an appropriate distance so that a shallow tissue such as a region of the tonsil can be inspected.

Moreover, an ultrasonic probe is desired, which has a body structure that allows a head unit to be easily brought into contact with a narrow and curved region inside a cavity.

### SUMMARY

An ultrasonic cavity probe, according to some embodiments of the present disclosure, includes a grip configured to be held by a user, a lens unit having a predetermined curvature radius, and configured to be inserted into a bodily cavity and to be brought into contact with a skin inside the bodily cavity, a head unit including a first side for mounting the lens unit and rounded corners, and a connecting portion configured to connect the head unit and the grip and to make a first angle with the head unit.

An ultrasonic medical device, according to some embodiments of the present disclosure, includes an ultrasonic probe having the above-mentioned structure.

### Advantageous Effects

The ultrasonic cavity probe according to the above-mentioned embodiments has a small head unit and a small cross-sectional area and a long length of a connecting portion, and hence it is easy to insert the ultrasonic probe into a cavity or mouth, allowing a tonsil or a sublingual region to be directly inspected.

Further, the ultrasonic probe includes a small and convex or concave lens unit, and hence it is easy to bring the ultrasonic probe into contact with a narrow and curved site such as a cavity or oral region of the tonsil or a sublingual region, thus providing an efficient inspection of the tonsil or the like.

The lens unit according to the above-mentioned embodiments has a small curvature radius, such that the focal length is short, and hence a shallow tissue such as the tonsil can be efficiently inspected.

### BRIEF DESCRIPTION OF DRAWINGS

FIG. 1 is a schematic diagram of an internal structure of a cavity represented by a mouth.

FIG. 2 is a perspective view of an ultrasonic cavity probe according to some embodiments of the present disclosure.

FIG. 3 is a schematic diagram of an ultrasonic generating unit mounted inside an ultrasonic probe according to some embodiments of the present disclosure.

FIG. 4 is a cross-sectional view of a head unit of an ultrasonic probe according to some embodiments of the present disclosure.

FIG. 5 is a side view of an ultrasonic probe according to some embodiments of the present disclosure.

FIGS. 6A and 6B are schematic diagrams of a connecting portion and a grip connected to each other via a hinge structure according to some embodiments of the present disclosure.

FIG. 7 is a schematic diagram of a connecting portion formed in an accordion structure according to some embodiments of the present disclosure.

REFERENCE NUMERALS

| | | | |
|---|---|---|---|
| 1: | Tonsil | 2: | Sublingual region |
| 10: | Ultrasonic cavity probe | 20: | Head unit |
| 30: | Ultrasonic generating unit | 32: | Lens unit |
| 40: | Grip | 41: | Concave portion |
| 42: | Groove portion | 43: | Gyro sensor |
| 50: | Connecting portion | | |

### DETAILED DESCRIPTION

Hereinafter, at least one embodiment of the present disclosure will be described in detail with reference to the accompanying drawings. In the following description, like reference numerals designate like elements although the elements are shown in different drawings. Further, in the following description of the at least one embodiment, a detailed description of known functions and configurations incorporated herein will be omitted for the purpose of clarity and for brevity.

Additionally, in describing the components of the present disclosure, various terms such as first, second, A, B, (a), (b), etc., are used solely for the purpose of differentiating one component from another, and one of ordinary skill would understand the terms are the other but not to imply or suggest the substances, order or sequence of the components. If a component were described as ‘connected’, ‘coupled’, or ‘linked’ to another component, one of ordinary skill in the art would understand they may mean the components are not necessarily only directly ‘connected’, ‘coupled’, or ‘linked’ but also are indirectly ‘connected’, ‘coupled’, or ‘linked’ via one or more additional components a third component.

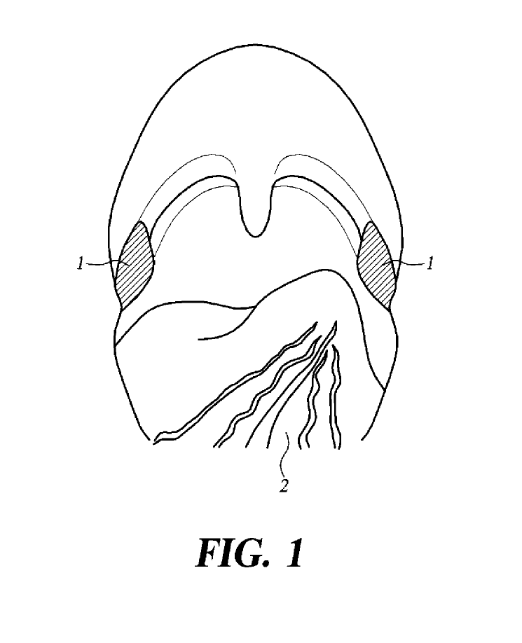

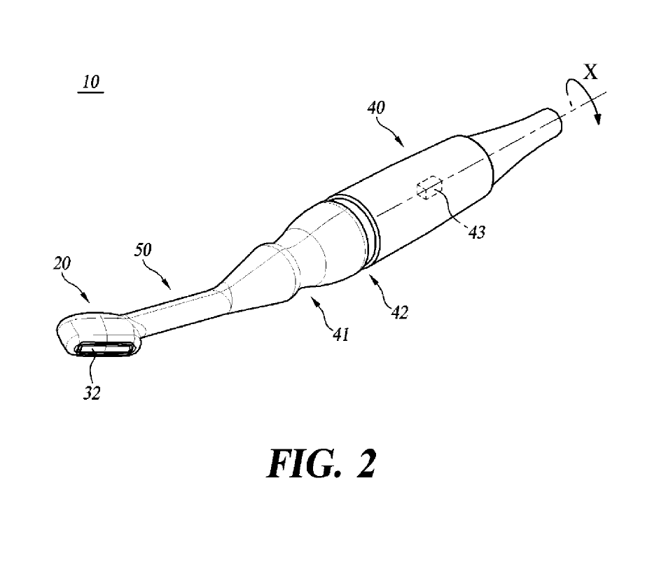

FIG. 1 is a schematic diagram of an internal structure of a mouth. FIG. 2 is a perspective view of an ultrasonic cavity probe 10 according to some embodiments of the present disclosure.

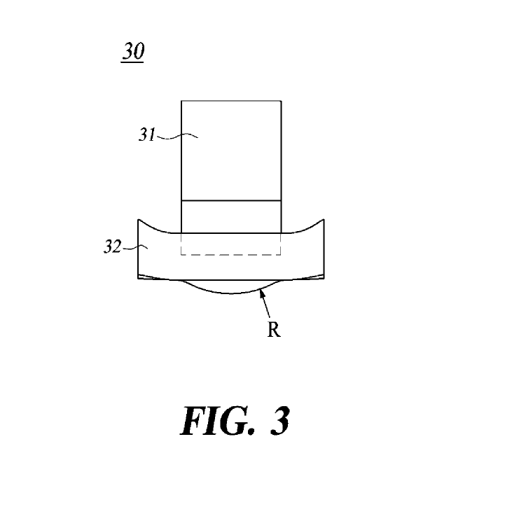

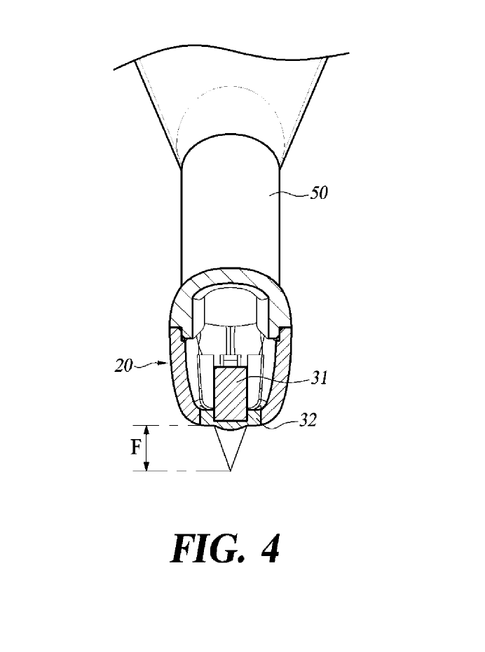

FIG. 3 is a schematic diagram of an ultrasonic generating unit 30 mounted inside the ultrasonic probe 10 according to some embodiments of the present disclosure. FIG. 4 is a cross-sectional view of a head unit 20 of the ultrasonic probe 10 according to some embodiments of the present disclosure.

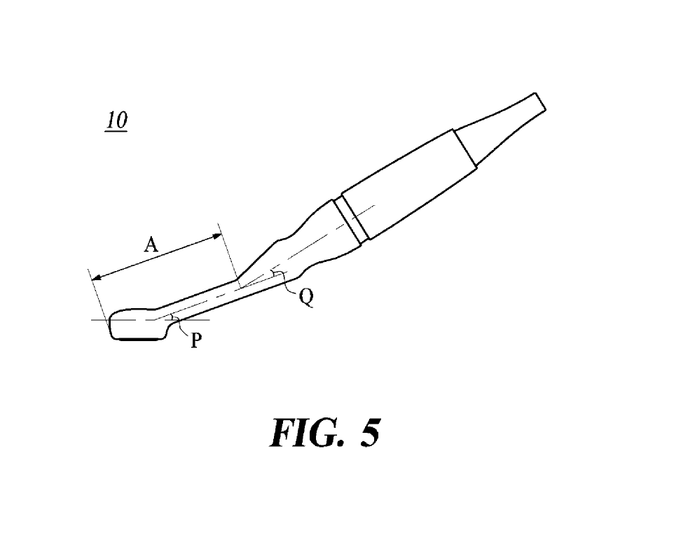

FIG. 5 is a side view of the ultrasonic probe 10 according to some embodiments of the present disclosure.

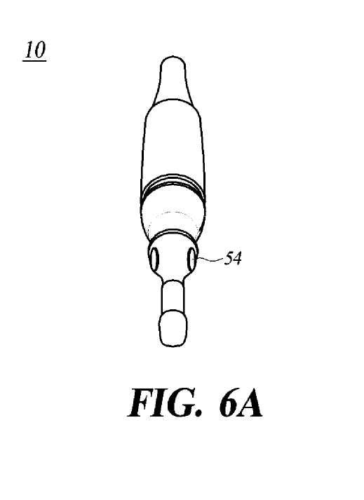

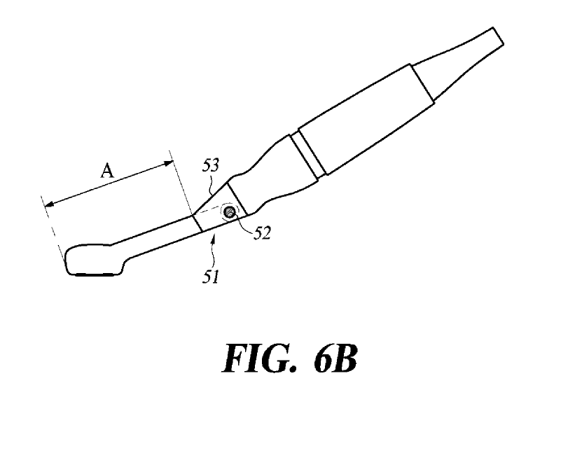

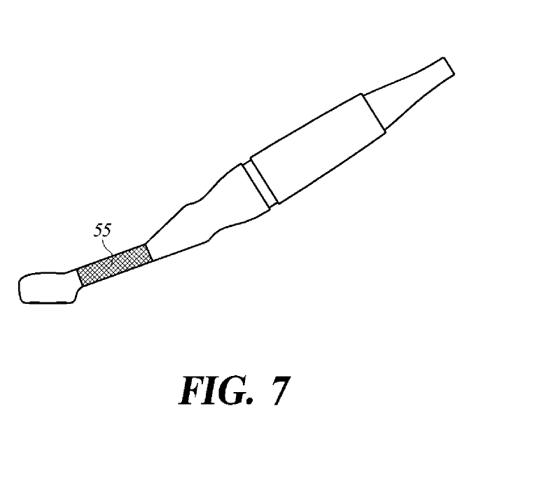

FIG. 6 is a schematic diagram of a connecting portion 50 and a grip 40 connected to each other via a hinge structure according to some embodiments of the present disclosure. FIG. 7 is a schematic diagram of the connecting portion 50 formed in an accordion structure 55 according to some embodiments of the present disclosure.

The ultrasonic cavity probe 10 according to some embodiments of the present disclosure may include the head unit 20, the connecting portion 50 and the grip 40.

In some embodiments, the head unit 20 includes a lens unit 32 on one surface thereof. The head unit 20 has rounded corners. The head unit 20 is a portion that is inserted into a cavity. Therefore, if the corners are angulated, the head unit 20 may cause damage inside the cavity when being inserted. However, as the corners of the head unit 20 according to some embodiments are rounded, the inside of the cavity can be prevented from being damaged by the head unit 20 when the head unit 20 is inserted into the cavity.

The inside of the head unit 20 includes the ultrasonic generating unit 30. In some embodiments, the ultrasonic generating unit 30 includes a layered member 31 and the lens unit 32. In some embodiments, the layered member 31 includes an acoustic absorption layer, a printed circuit board, a piezoelectric plate, a ground layer, and an acoustic matching layer, layered in this order.

The lens unit 32 can be formed in an integrated manner at a position where the acoustic matching layer is located. The lens unit 32 serves to focus the ultrasonic wave generated from the piezoelectric plate.

In some embodiments, the lens unit 32 includes a convex lens having a convex front plane. In some embodiments, the lens unit 32 includes a concave lens having a concave front plane. In some embodiments, the lens unit 32 includes a planar lens having a planar front plane.

The first among the features of some embodiments is that the head unit 20 includes the lens unit 32 having a small convex front plane. The small convex front plane of the lens unit 32 can be easily brought into contact with a narrow and curved skin inside the cavity, and hence it can be advantageously adapted to an otolaryngology treatment, particularly in examining a tonsil 1 or a sublingual region 2 that is the bottom portion of a tongue.

A second feature of some embodiments is that the head unit 20 includes the lens unit 32 having a small curvature radius R. In other words, the head unit 20 according to some embodiments includes the lens unit 32 on one surface thereof, and the lens unit 32 can be insertably brought into contact with the skin inside the cavity, having the curvature radius R of a predetermined magnitude. In some embodiments, the curvature radius R is 1 millimeter to 20 millimeters. As the curvature radius R is small, a sharp and accurate image can be obtained in an otolaryngology case, particularly in checking the tonsil 1 or the sublingual region 2.

In some embodiments, the lens unit 32 has a predetermined focal length F such that the lens unit 32 can be inserted into the cavity, is brought into contact with the skin inside the cavity, and focuses the ultrasonic wave on the inside of the skin. In some embodiments, the focal length F is 3 millimeters to 20 millimeters.

One of the many features of some embodiments is that the lens unit 32 has a short focal length F.

When the focal length F is too long, it may be hard to inspect a thin portion like the tonsil 1. This problem can be solved by adopting the lens unit 32 having the short focal length F as in some embodiments of the present disclosure.

The ultrasonic probe 10 according to some embodiments of the present disclosure includes the grip 40 for a user to hold the ultrasonic probe 10 by hand. Further, the ultrasonic probe 10 includes the head unit 20 and the connecting portion 50 that connects the head unit 20 and the grip 40 and makes a first angle P relative to the head unit 20. In some embodiments, the first angle P is 10 degrees to 50 degrees. As the head unit 20 and the connecting portion 50 maintain a predetermined angle, a user can conveniently bring the lens unit 32 of the ultrasonic probe 10 into contact with the narrow and curved skin inside the cavity.

With the ultrasonic probe 10 according to some embodiments of the present disclosure, the head unit 20 and at least a portion of the connecting portion 50 can be inserted into the cavity, and the region on the inner side of the skin can be observed by generating the ultrasonic wave in a direction toward the inner side of the skin inside the cavity. To this end, the head unit 20 has a small size to move freely inside the cavity. Further, the connecting portion 50 has a small cross-sectional area and is formed in an elongated manner in the longitudinal direction. In some embodiments, a length A that is insertable into the cavity including the head unit 20 and the connecting portion 50 is 50 millimeters to 80 millimeters. In some embodiments, the length A is from the end of the head unit 20 and extends to a point where the connecting portion 50 meets the grip 40. In some embodiments, when the ultrasonic probe 10 includes a hinge coupling structure 51, which is described later, the length A is from the end of the head unit 20 and extends to a point where the head unit 20 meets a hinge coupling cover 53.

A linear probe according to conventional ultrasonic probe technologies has too large a head unit to insert it into the cavity, and hence a user has to bring the head unit of the probe into contact with the skin outside the chin of a patient in order to observe the tonsil 1 or the like. However, this can hardly provide an accurate and sharp image.

The ultrasonic probe 10 according to some embodiments of the present disclosure has the head unit 20 having a small size and the connecting portion 50 having a small cross-sectional area and a long length in the longitudinal direction, and hence the ultrasonic probe 10 can be inserted into the cavity and be brought into contact with the region of the tonsil 1 directly inside the cavity, which enables an accurate and sharp image to be obtained.

The grip 40 of the ultrasonic probe 10 according to some embodiments of the present disclosure has a cross-sectional area of a size appropriate to be held by the hand of a user. Further, in some embodiments, the grip 40 includes a concave portion 41 along the circumferential surface of the grip 40 on which a thumb of the user can rest while rotating the grip 40 in the axial direction (X direction) of the grip 40. In the ultrasonic probe technology, the concave portion 41 for placing the thumb of the user is formed only at a part of the circumferential surface of the grip, and hence the user has to twist his or her wrist when moving the head unit of the probe to the left or the right, causing an inconvenience in using the probe. However, in the ultrasonic probe 10 according to some embodiments, the concave portion 41 is formed along the circumferential surface, and hence the grip 40 can be rotated in the axial direction (X direction) of the grip 40 without twisting the wrist, which is convenient. The ultrasonic probe 10 according to some embodiments of the present disclosure includes a groove portion 42 which is formed along the circumferential surface of the grip 40, for fixing a sheath. Alternatively, the sheath can be fixed to the concave portion 41. The sheath is a sort of cover for covering the head unit 20 and the connecting portion 50 of the ultrasonic probe 10 for sanitation when a user performs a diagnosis by using the ultrasonic probe 10. As the groove portion 42 allows the user to easily fix the sheath to the grip 40, the user can conveniently use the ultrasonic probe 10. The sanitation factor is well incorporated into the ultrasonic probe 10 according to some embodiments as it is an insertion type for bodily cavities. Using the sheath, the ultrasonic probe 10 saves repeated cleaning sessions to further the user convenience without compromising the sanitation.

In the ultrasonic probe 10 according to some embodiments of the present disclosure, the connecting portion 50 and the grip 40 make a second angle Q. In some embodiments, the second angle Q is 1 degree to 40 degrees. As the connecting portion 50 and the grip 40 make a predetermined angle, the user can bring the lens unit 32 of the ultrasonic probe 10 into contact with a cavity portion that is narrow and hard to reach in a more convenient manner.

In some embodiments, the connecting portion 50 and the grip 40 are connected to each other via a hinge (see FIG. 6). This hinge coupling allows the second angle Q to be adjusted to a predetermined angle. Further, in some embodiments, the ultrasonic probe 10 includes an angle adjustment button 54 at a portion where the connecting portion 50 and the grip 40 are hinge-coupled, to adjust the second angle Q to the predetermined angle. In some embodiments, the hinge coupling structure 51 includes a first hinge coupling portion that is extended from the connecting portion 50, a second hinge coupling portion included in the grip 40, and a hinge axis 52 that hinge-couples the first hinge coupling portion and the second hinge coupling portion. The hinge coupling cover 53 is made of soft material such as silicone and covers the hinge-coupled portion. The angle adjustment button 54 has a typical structure, and hence a detailed description thereof is omitted.

In some embodiments, at least a portion of the connecting portion 50 includes the accordion structure 55 (see FIG. 7). The accordion structure 55 allows a user to bend the connecting portion 50 to a desired shape. The accordion structure 55 has rigidity. Therefore, the user can bring the lens unit 32 into contact with a desired region in the cavity while maintaining the bent shape of the connecting portion 50.

The ultrasonic cavity probe 10 according to some embodiments of the present disclosure includes the grip 40 that can be held by a hand of a user. The ultrasonic probe 10 further includes the lens unit 32 that is inserted into the cavity and brought into contact with the skin inside the cavity, and has the predetermined focal length F to allow the ultrasonic wave generated from the piezoelectric plate to be focused on the inside of the skin. The ultrasonic probe 10 further includes the head unit 20 including the lens unit 32 on one surface thereof. The ultrasonic probe 10 further includes the connecting portion 50 that connects the head unit 20 and the grip 40, and makes the first angle P with the head unit 20 and the second angle Q with the grip 40.

In some embodiments, the focal length F is 3 millimeters to 20 millimeters. For example, the ultrasonic probe 10 according to some embodiments of the present disclosure includes the lens unit 32 having the focal length F of 5 millimeters. If the focal length F is too long, it becomes hard to inspect a shallow portion such as the tonsil 1. This problem can be solved by adopting the lens unit 32 having the short focal length F.

In some embodiments, the first angle P is 10 degrees to 50 degrees, and the second angle Q is 1 degree to 40 degrees. The second angle Q can be adjusted to a predetermined angle. A structure for adjusting the angle is a hinge structure in some embodiments and the accordion structure 55 in other embodiments.

The ultrasonic cavity probe 10 according to some embodiments of the present disclosure further includes a gyro sensor unit 43 for sensing the orientation of the head unit 20. The gyro sensor unit 43 allows the user of the ultrasonic probe 10 to easily check, for example, whether a region under inspection or image information acquired and recorded by the ultrasonic probe 10 is the tonsil 1 on the right or left side.

The present disclosure in some embodiments provides an ultrasonic medical device including the above-mentioned ultrasonic probe 10. The ultrasonic medical device may be of any type so long as the ultrasonic probe 10 is mountable thereon.

Although exemplary embodiments of the present disclosure have been described for illustrative purposes, those skilled in the art will appreciate that various modifications, additions and substitutions are possible, without departing from the spirit and scope of the claimed disclosure.

1. An ultrasonic cavity probe, comprising:
a hand-held grip;
a lens unit having a predetermined curvature radius, and configured to be inserted into a bodily cavity and to be brought into contact with a skin inside the bodily cavity;
a head unit including
a first side for mounting the lens unit, and
rounded corners; and
a connecting portion configured to connect the head unit and the grip and to make a first angle with the head unit.

2. The ultrasonic cavity probe according to claim 1, wherein the curvature radius is in a range from 1 millimeter to 20 millimeters.

3. The ultrasonic cavity probe according to claim 1, wherein the first angle is in a range from 10 degrees to 50 degrees.

4. The ultrasonic cavity probe according to claim 1, wherein the connecting portion and the grip make a second angle.

5. The ultrasonic cavity probe according to claim 4, wherein the second angle is in a range from 1 degree to 40 degrees.

6. The ultrasonic cavity probe according to claim 4, further comprising a hinge configured to connect the connecting portion and the grip and to adjust the second angle to a predetermined angle.

7. The ultrasonic cavity probe according to claim 6, further comprising an angle adjustment button on the hinge that connects the connecting portion and the grip and to adjust and maintain the second angle to the predetermined angle.

8. The ultrasonic cavity probe according to claim 1, wherein at least a portion of the connecting portion has a rigid accordion structure.

9. The ultrasonic cavity probe according to claim 1, wherein a length including the head unit and the connecting portion is in a range from 50 millimeters to 80 millimeters.

10. The ultrasonic cavity probe according to claim 1, wherein the lens unit includes any one selected from the group consisting of a convex lens, a concave lens and a planar lens.

11. The ultrasonic cavity probe according to claim 1, wherein the grip includes a concave portion along a circumferential surface of the grip, on which a user's thumb rests while rotating the grip in an axial direction of the grip by hand.

12. The ultrasonic cavity probe according to claim 1, wherein the grip includes a concave portion along a circumferential surface of the grip, on which a sheath is fixable.

13. The ultrasonic cavity probe according to claim 1, wherein the grip includes a groove portion along a circumferential surface of the grip, on which a sheath is fixable.

14. The ultrasonic cavity probe according to claim 1, wherein the lens unit has a predetermined focal length that allows the lens unit to be inserted into the bodily cavity, to be brought into contact with the skin inside the bodily cavity, and to focus an ultrasonic wave inside the skin.

15. The ultrasonic cavity probe according to claim 14, wherein the focal length is in a range from 3 millimeters to 20 millimeters.

16. The ultrasonic cavity probe according to claim 1, further comprising a gyro sensor unit configured to detect an orientation of the head unit inside the bodily cavity.

17. An ultrasonic medical device, comprising the ultrasonic cavity probe according to claim 1.
